(19) United States
(12) Plant Patent Application Publication
(10) Pub. No.: US 2009/0210972 P1
(43) Pub. Date: Aug. 20, 2009

(54) CHESTNUT PLANT NAMED 'AU BUCK II'

(50) ;

(75) Inventors: W. Alfred Dozier, JR., Opelika, AL (US); J. D. Norton, Opelika, AL (US); Curtis J. Hansen, Opelika, AL (US)

Correspondence Address: HAVERSTOCK & OWENS LLP, 162 N WOLFE ROAD, SUNNYVALE, CA, 94086, US

(73) Assignee: Auburn University, an Alabama Corporation

(21) Appl. No.: 12/012,025

(22) Filed: Jan. 30, 2008

(51) Int. Cl. A01H 5/00

(52) U.S. Cl. PLT/152

(57) ABSTRACT

‘AU Buck II’ is a new and distinct Chinese chestnut cultivar that is blight resistant, precocious and prolific. It produces a large nut. Nut drop begins about September 15 and continues for a 6-7 week period. A large percentage of the nuts drop during the first three weeks after the beginning of nut drop. ‘AU Buck II’ nuts mature and start dropping during the early phase of the normal ripening period that most Chinese chestnut cultivars mature and drop nuts in the area. ‘AU Buck II’ is a large tree that was 9.14 meters tall, with a canopy area of 101.36 square meters at 15-years of age. ‘AU Buck II’ is the second cultivar to mature and drop nuts in a series of four Chinese chestnut cultivars that will provide for a continuous nut drop of a high energy wildlife food source from late August through mid-to-late November.

### LATIN NAME OF THE GENUS AND SPECIES OF THE PLANT CLAIMED

Castanea P. Mill., Castanea mollissima Blume.

### VARIETY DENOMINATION

‘AU BUCK II’

### BACKGROUND OF THE INVENTION

A Chinese chestnut planting was established at Auburn University, Auburn, Ala., from nuts collected in China. The planting was established on the United States Department of Agriculture Horticulture Farm which in later years became the Mainstation Horticulture Farm. Precocious and prolific-bearing, blight resistant seedlings were selected for nut appearance, size and quality. Each generation of seedlings were the product of controlled mass pollination from the most promising seedlings selected from the previous generation. ‘AU Cropper’, ‘AU Leader’ and ‘AU Homestead’ were released from a second generation of approximately 2000 seedlings. A planting of third generation seedlings from controlled mass pollination of ‘AU Leader’, ‘AU Homestead’ and ‘AU Cropper’ was established at the Auburn University Piedmont Substation at Camp Hill, Ala.

### SUMMARY OF THE INVENTION

‘AU Buck II’ is an open pollinated seedling of ‘AU Homestead’.

The present invention relates to a new and distinct Chinese chestnut cultivar that is blight resistant, precocious, produces large sized nuts (16.6 g), and begins nut drop about September 15 and continues for a 6-7 week period. A large percent of the nuts drop during the first three weeks after nut drop begins. The nuts mature and start dropping during the early part of the normal ripening period for most Chinese chestnut cultivars in this area. The large crop of large sized nuts is an excellent high energy food source for wildlife such as deer and squirrels. The original 15-year old ‘AU Buck II’ tree is a large tree, 9.14 meters tall, with a canopy width of 11.35 meters, and a canopy area of 101.36 square meters. The tree has a trunk diameter of 37.9 cm at breast height. The ‘AU Buck II’ produced nuts the third year after transplanting, has produced a large crop annually for the size of tree, and produced 127.9 kg (282 pounds) of nuts in 2006.

‘AU Buck II’ cultivar is the second cultivar to mature and drop nuts in a series of four Chinese chestnut cultivars that will provide a continuous nut drop of a high energy wildlife food source from late August through mid-to-late November. ‘AU Buck I’, ‘AU Buck III’ and ‘AU Buck IV’ are the other three cultivars, which are disclosed in U.S. patent application Ser. No. ______, filed on ______, and entitled “CHESTNUT PLANT NAMED ‘AU BUCK I’” [Attorney Docket No. AUB-07100], U.S. patent application Ser. No. ______, filed on ______, and entitled “CHESTNUT PLANT NAMED ‘AU BUCK III’” [Attorney Docket No. AUB-07300] and U.S. patent application Ser. No. ______, filed on ______, and entitled “CHESTNUT PLANT NAMED ‘AU BUCK IV’” [Attorney Docket No. AUB-07400], which are hereby incorporated by reference.

The new cultivar is able to be asexually reproduced by budding or grafting onto a seedling Chinese chestnut rootstock. The unique characteristics come true to form and are established and transmitted through asexual propagation.

### BRIEF DESCRIPTION OF THE DRAWINGS

FIG. 1 is a photograph of nuts of the ‘AU BUCK II’ cultivar.

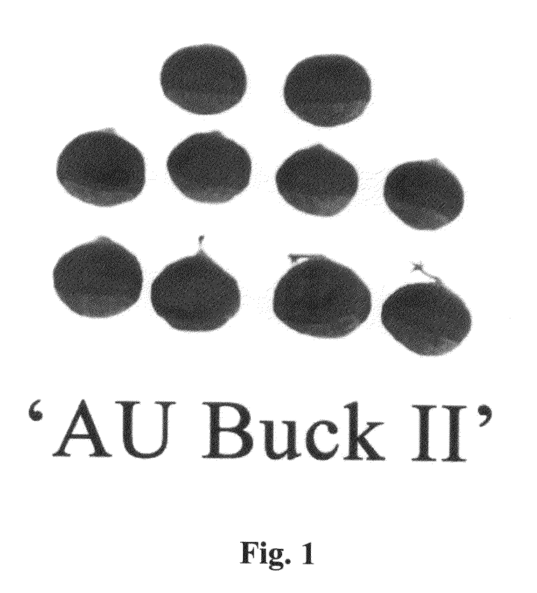

FIG. 2 is a photograph of nuts of the ‘AU BUCK II’ cultivar.

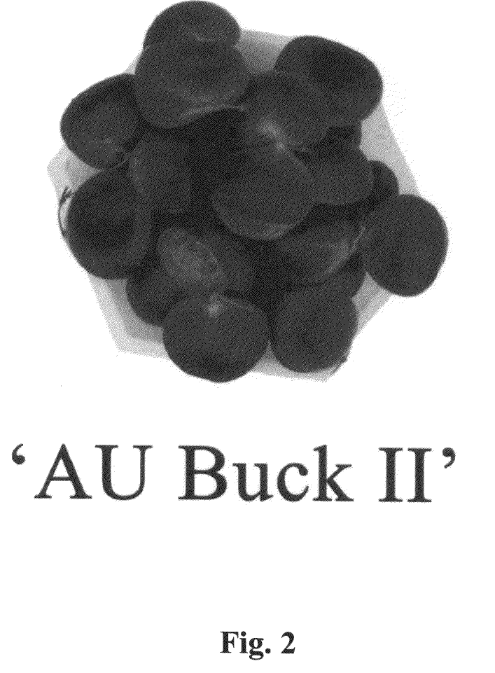

FIG. 3 is a photograph of a tree of the ‘AU BUCK II’ cultivar.

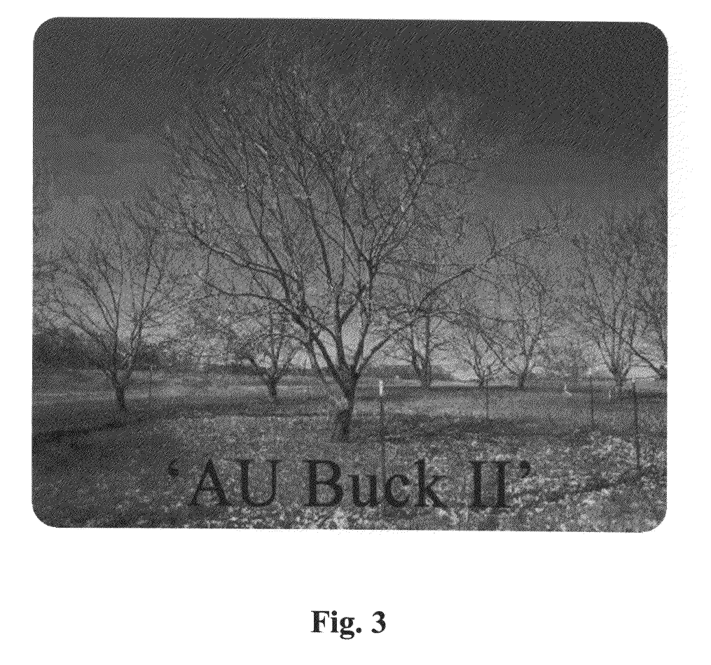

### DETAILED BOTANICAL DESCRIPTION

The Chinese chestnut, Castanea mollissima Blume, is a cold, hardy, temperate zone species native to China. It can be grown between 30° and 50° latitudes. The Chinese chestnut is resistant to chestnut blight fungus Cryphonectria parasitica. Generally, Chinese chestnuts are grown on a wide range of soils, but well-drained, deep and fertile soils are considered the best. Soils should be slightly acidic with pH 5.6-6.5. The name mollissima means soft hair and this species is recognized by dense hair on young leaves and downy yellow terminal parts of the shoots in winter. The leaf blade is thicker, and, in general, mature leaves are broader than those of other species. The nuts have a small scar or hilum. The pellicle or thin membranous skin on the nuts is thin and peels readily from the kernel. The trees are a spreading type and long-lived with a round top. The trees have bark with furrows and buds with 3-4 scales and leaves are 2 ranked, serrated with numerous parallel veins.

Chestnuts are monoecious and staminate flowers appear on erect cylindrical catkins with 10-20 stamens and 6-parted calyx. Pistillate flowers are borne on a lower part of the upper staminate catkins and rarely on separate catkins usually 3 in a prickly symmetrical involucre with 7-9 styles and a 6-celled ovary. Nuts are small, brown with a pale scar at the base. Generally, 1-3 nuts per involucre or bur are present. ‘AU Buck II’ is a tall tree that is obovate in shape with high, diffuse and upright branches. The original 15-year old tree is 9.14 meters (29.96 feet) tall with a trunk diameter at breast height of 37.90 cm (14.92 inches). The canopy is 11.35 meters (37.20 feet) wide and covers an area of 101.36 square meters (1091.0 square feet). The average nut length is 30.70 mm (1.21 inches) and the average nut width is 37.70 mm (1.48 inches). The nuts start dropping about September 15 and continue to drop for a 6 to 7 week period. A large percent of the nuts drop during the first three weeks after nut drop begins. The nuts mature and start dropping during the early part of the normal ripening for most Chinese chestnuts cultivars in this area. ‘AU Buck II’ is a precocious and prolific fruiting cultivar.

In the planting at the Piedmont Substation, Camp Hill, Ala., accurate yields could not be obtained due to extremely heavy wildlife (deer and turkey) feeding. Therefore, the trees were rated for crop load each year. ‘AU Buck II’ has been a producer of a large crop load each season. In the fall of 2006, individual trees were caged with 6 foot (1.83 meters) tall chicken wire prior to nut drop to exclude wildlife and nuts were picked up daily during nut drop. The original 15-year old ‘AU Buck II’ tree produced 127.9 kg (282.0 pounds) of nuts in 2006. This is a much greater yield than the yields of similar aged trees.

The table below illustrates the specific differences between the ‘AU BUCK II’ cultivar and the ‘REVIVAL’ cultivar.

The botanical details of this new and distinctive variety of
chestnut tree-with color definitions (except those in
common color terms) referenced to Royal Horticultural
Society's Colour Chart (RHS) and color was also determined
using an electronic spectrophotometer to determine hue angle
and Chroma (spectrophotometer model CM-2002; Minolta
Camera Co., Japan).

‘AU BUCK II’ CHESTNUT
Tree:
Size (at maturity)-large-fifteen year old original tree is 9.14 meters tall
with a canopy width of 11.35 meters and a canopy area of
101.36 sq. meters Tree shape is round.
Vigor-very vigorous
Trunk:
Form-upright with branches low and diffuse
Texture-relatively smooth
Color of bark-brown RHS N200B, Chroma C* 11.85, hue angle 85.23
Branches:
Form-upright and spreading
Texture-relatively smooth
Lenticels-few, small
Branching habit-low and spreading
Color-new wood: brown, RHS 200C, Chroma C* 13.80,
hue angle 71.55, mature wood: grey-brown RHS N199A
Chroma C* 13.69, hue angle 91.59
Foliage:
Quantity-abundant
Density-dense
Leaves:
Size-large. Length (cm) 20.5 (17.5-23.5) [20]
width (cm) 8.2 (7.2-9.3) [20]
leaf ratio 2.5 (2.1-3.0) [20]
Shape-oblong-elliptic to obovate
leaf tip-acuminate to acute
leaf base-rounded; equal to oblique
Thickness-thick. Leaf venation 1° pinnate: 2° ± parallel,
prominent abaxially
Texture-moderately coriaceous
Margin-weakly to coarsely serrate; teeth ascending
Petiole-length 0.9 cm (0.5-1.8) [20]
Petiole pubescence-sparingly to generously pubescence of simple hairs
Color-adaxial surface, glabrous blade; scattered simple hairs on main veins
medium green; moderately shiny, RHS 147A,
Chroma C* 14.62, hue angle 120.75
abaxial surface, moderately to densely stellate pubescence on blade;
simple hairs along main veins, RHS 147B, Chroma C* 18.44,
hue angle 110.70
Bloom:
Amount of bloom-heavy
Color-at anthesis, 161D greyed-yellow group, 157D green-white group,
155C white group
Blooming period-mid-May. After foliation in April.
Age at which tree starts flowering-early, 2-3 years alter graft
replacement.
Male flower-Catkin length (cm)-13.7 (11.8-15.7) [15]
Male flower-stamen number per catkin-11.3 (10-12) [15]
Female flower-flower number per bur-3
Female flower-style number per flower 6.7 (5-8) [12]
Crop:
Bearing-regular annual bearer
Productivity-prolific
Ripening period-long, nuts begins dropping September 14 and
continues for a 4-5 week period.
Distribution of nuts on tree-well distributed, fruits on terminals with
2-4 burs per terminal.
Tenacity-burs crack while on tree and nuts release and drop from bur.
Hull:
Description-spiny round bur
Size-2.98-3.71″ in diameter
Number of nuts-2-3 per bur
Dehiscence-splits easily when still on tree
Color-yellow-green at dehiscence, RHS N144C
Nut:
Size-very large; average size- 1.21″ × 1.48 × 0.81″.
Average weight-16.6 grams 27 (23-33) nuts per pound.
Form-very large, broader than long, flattened on 1 side, occ. 2, mostly
hemispheric on other side; rounded basally, almost flat distally with little
or no tip.
Blossom end-small pointed tip
Basal end-flattened
Color-light brown, RHS 200B, Chroma C* 14.03, hue angle 40.05
Pubescence-mixed long and short, fine, white hairs densely
covering the ⅛ to ¼ end, otherwise sparse and glabrate elsewhere
Shell-thin
Hardness of shell-relatively hard, yet not rigid
Texture of shell-smooth
Percentage of kernel to nut-very high-90-95%
Kernel:
Size-almost as large as nut size
Form-same as nut shape
Pellicle-thin
Flavor-excellent, very sweet
Color-straw color, RHS 152D, Chroma C* 45.86, hue angle 81.35
Resistance to insects: no unusual susceptibilities noted
Resistance to disease: no susceptibilities to disease noted
The chestnut tree and its nuts herein described may
vary in slight detail due to climatic and soil conditions
under which the variety may be grown; the present
description being of the variety as grown in Camp Hill,
Ala.

The botanical details of this variety of
chestnut tree-with color definitions (except those in
common color terms) referenced to Maerz and Paul
Dictionary of Color-are as follows:

‘REVIVAL’
Tree:
Size (at maturity)-large
Vigor-very vigorous
Trunk:
Form-upright with branches spreading in upper
reaches of tree.
Texture-relatively smooth
Color of bark-Silvergray (13-A-1)
Branches:
Form-strong
Texture-relatively smooth
Lenticels-few, small
Branching habit-spreading in upper region of tree
Color-new wood: reddish brown and glossy, mature
wood: silver gray
Foliage:
Quantity-abundant
Density-dense
Leaves:
Size-large. Average length-5-7″ (including petiole).
Average width-2″
Shape-oblong with acute tip and rounded base
Thickness-thick
Texture-smooth
Margin-dentate
Petiole-length: medium. Thickness: medium.
Color-Top side-glossy dark green (22-L-12).
Under side-lighter green (21-D-7).
Bloom:
Amount of bloom-heavy
Color-cream white (17-B-1)
Blooming period-late. After leaf out in April
Age at which tree starts flowering-early; 2-3 years
years after graft replacement.
Crop:
Bearing-regular (yearly) bearer
Productivity-prolific
Ripening period-short. September 15-October 1.
Distribution of nuts on tree-well distributed
Tenacity-burrs crack while on tree and nuts easily
release, many falling by themselves
Hull:
Description-spiny, round burr
Size-3-4″ in diameter
Number of nuts-2-3 per burr
Dehiscence-splits easily when still on tree. Some
entire burrs split and fall to ground
Color-brown (15-A-8)
Nut:
Size-large. Average size-1⅛″X1⅛″X1″ thick.
Average weight-24-32 nuts per pound
Form-broad and ovoid on one side, flat on other side
Blossom end-pointed tip
Basal end-flattened
Color-India Red (7-L-6).
Shell-thin
Hardness of shell-relatively hard, yet not rigid
Texture of shell-smooth
Percentage of kernel to nut-very high (95%)
Kernel:
Size-almost as large as nut size
Form-same as nut shape
Pellicle-thin
Flavor-excellent. Very sweet.
Color-Oyster white (10-B-1)
Resistance to insects: no unusual susceptibilities noted
Resistance to disease: very high inherent resistance to
chestnut bark fungus (Endothia parastica), no
other susceptibilities to any other disease
The chestnut tree and its nuts herein described may
vary in slight detail due to climatic and soil conditions
under which the variety may be grown; the present
description being of the variety as grown in Alachua,
Fla.

What is claimed is:

1. A new and distinct cultivar of the species Castanea mollissima Blume named ‘AU BUCK II’ as described and illustrated herein.
